SEQUENCE LISTING (11 sequences)

SEQ ID NO: 1 - DNA, 30 bp, Artificial Sequence
  Description of Artificial Sequence Synthetic primer
agagcttgaggagagcaggaaaggtggaac

SEQ ID NO: 2 - DNA, 30 bp, Artificial Sequence
  Description of Artificial Sequence Synthetic primer
gggaggggaggtacagggttgaggctagtg

SEQ ID NO: 3 - DNA, 30 bp, Artificial Sequence
  Description of Artificial Sequence Synthetic primer
acaagggctactggttgccgatttttattg

SEQ ID NO: 4 - DNA, 27 bp, Artificial Sequence
  Description of Artificial Sequence Synthetic primer
gggactggcctcagaggaaacttcagg

SEQ ID NO: 5 - DNA, 27 bp, Artificial Sequence
  Description of Artificial Sequence Synthetic primer
gtttgagcaactctcaccattatgggc

SEQ ID NO: 6 - DNA, 22 bp, Artificial Sequence
  Description of Artificial Sequence Synthetic primer
cctgagggctcccagagagtgg

SEQ ID NO: 7 - DNA, 22 bp, Artificial Sequence
  Description of Artificial Sequence Synthetic primer
ggtttagcacgaccacaacagc

SEQ ID NO: 8 - DNA, 24 bp, Artificial Sequence
  Description of Artificial Sequence Synthetic primer
agcctagctccagtgcttctagta

SEQ ID NO: 9 - DNA, 23 bp, Artificial Sequence
  Description of Artificial Sequence Synthetic primer
ctttggatgaggagccgatattg

SEQ ID NO: 10 - DNA, 20 bp, Artificial Sequence
  Description of Artificial Sequence Synthetic primer
gatggatcatggctgacttc

SEQ ID NO: 11 - DNA, 20 bp, Artificial Sequence
  Description of Artificial Sequence Synthetic primer
agcaagcaactgactgctct

(19) United States
(12) Patent Application Publication
(10) Pub. No.: US 2008/0064071 A1
(43) Pub. Date: Mar. 13, 2008

(54) Zwitterionic detergents for the storage and use of DNA polymerases

(76) Inventors: Holly H. Hogrefe, San Diego, CA (US); Jeffrey Fox, Escondido, CA (US); Michael Borns, Escondido, CA (US); Jeffrey C. Braman, Carlsbad, CA (US)

Correspondence Address: AGILENT TECHOLOGIES INC, P.O BOX 7599, BLDG E , LEGAL, LOVELAND, CO, 80537-0599, US

(21) Appl. No.: 11/828,107

(22) Filed: Jul. 25, 2007

Related U.S. Application Data
(60) Provisional application No. 60/833,331, filed on Jul. 25, 2006.

(51) Int. Cl. C12P 19/34

(52) U.S. Cl. 435/91.2

(57) ABSTRACT

The present invention provides methods, compositions, and kits for storing and enhancing the activity of thermostable polymerases. The methods comprise mixing a thermostable polymerase with at least one zwitterionic detergent or non-detergent surfactant. Compositions and kits for performing the process according to the invention are also provided.

### CROSS-REFERENCE TO RELATED APPLICATIONS

This application relies on the disclosure and claims the benefit of the filing date of U.S. Application No. 60/833,331, filed on 25 Jul. 2006, the entire disclosure of which is incorporated herein by reference.

### BACKGROUND OF THE INVENTION

1. Field of the Invention

The present invention relates to the field of thermostable polymerases. More specifically, the present invention pertains to methods, compositions, and kits for stabilizing and enhancing the activity of thermostable polymerases.

2. Description of Related Art

Amplification of nucleic acids involves the thermal cycling of a reaction mixture containing a nucleic acid polymerase to generate an amplified target nucleic acid. An example of this thermal cycling process is that which occurs in Polymerase Chain Reaction (PCR), a laboratory technique that can theoretically take one molecule of DNA and produce measurable amounts of identical DNA in a short period of time. PCR is a widely used method in the fields of biotechnology, forensics, medicine, and genetic research. In this method, oligonucleotides are used as primers for a series of synthetic reactions that are catalyzed by a DNA polymerase. The reaction mixture is subjected to multiple cycles of denaturation, annealing, and synthesis performed at different temperatures. Thermostable polymerases are generally used to amplify the target nucleic acid sequences in these thermal cycling reactions because they are not inactivated by the heat denaturation step and, therefore, do not need to be replaced in every round of the amplification cycle. Although efficient, exponential amplification of target sequences is not an unlimited process. Under normal reaction conditions, the amount of DNA polymerase becomes limiting after a certain number of cycles of amplification.

Attempts have been made to improve the PCR amplification process by employing detergents and/or surfactants. For example, U.S. Pat. No. 6,127,155 discloses that the non-ionic detergents NP-40 and Tween stabilize Taq DNA polymerase. However, this patent does not disclose the use of non-detergent surfactants or zwitterionic detergents for the stability of thermostable polymerases in PCR reactions. As another example, U.S. Patent Application Publication No. 2003/0017567 discloses a method for performing an amplification reaction utilizing a dye that converts electromagnetic energy into thermal energy to heat the reaction mixture. A zwitterionic surfactant is added to the reaction mixture to reduce interference of the dye with the functioning of the nucleic acid polymerase. Additionally, U.S. Patent Application Publication No. 2002/0168658 discloses the use of zwitterions in combination with a compound that disrupts base pairing, e.g., DMSO, to improve the amplification of nucleic acids that are G+C rich. However, this publication does not disclose the use of zwitterionic detergents alone in improving the amplification of nucleic acids and actually teaches that the zwitterionic detergents used should be selected carefully so as not to inhibit the activity of the DNA polymerase in the reaction.

Given the widespread use and importance of thermal cycling processes, there is a need in the art for a way to improve the stability and/or enhance the activity of thermostable enzymes used in DNA amplification.

### SUMMARY OF THE INVENTION

The present invention provides compositions, kits, and methods that include a polymerase and a zwitterionic detergent or non-detergent surfactant. Specifically, such compositions, kits, and methods are useful in molecular biology techniques, such as PCR, Quantitative Real Time PCR (QPCR), sequencing, and mutagenesis. The present invention is based in part on the surprising finding that zwitterionic detergents and non-detergent surfactants increase stability and enhance activity of thermostable polymerases.

In a first aspect, the invention is directed to storage compositions. In one embodiment, the storage composition comprises at least one purified polymerase and at least one zwitterionic detergent or non-detergent surfactant. The composition may comprise two or more zwitterionic detergents as well as independently comprising two or more purified polymerases. In some embodiments, the storage composition does not contain a detectable label. In other embodiments, the invention is directed to a storage composition that includes a purified polymerase, a labeled nucleotide, and a zwitterionic detergent or non-detergent surfactant. In yet another embodiment, the invention is directed to a storage composition that includes a purified polymerase, a fluorescent DNA binding dye, and a zwitterionic detergent or non-detergent surfactant, wherein the fluorescent DNA binding dye produces a detectable signal when bound to a target nucleic acid, such as DNA.

In a second aspect, the invention provides reaction mixtures. In one embodiment, the invention is directed to a reaction mixture that includes at least one purified polymerase, at least one oligonucleotide probe, and at least one zwitterionic detergent or non-detergent surfactant. The composition may comprise two or more zwitterionic detergents or surfactants or independently two or more purified polymerases. A detectable label is operatively coupled to at least one of the oligonucleotide probes. In another embodiment, the invention comprises a reaction mixture having a purified polymerase, a labeled nucleotide, and a zwitterionic detergent or non-detergent surfactant. In yet another embodiment, the reaction mixture includes a purified polymerase, a fluorescent DNA binding dye, and a zwitterionic detergent or non-detergent surfactant, wherein the fluorescent DNA binding dye produces a detectable signal when bound to a target nucleic acid, such as DNA. In still another embodiment, the invention is directed to a reaction mixture that includes nucleoside-5′-triphosphates, primers, a buffer in which primer extension can occur, a polymerase, an oligonucleotide probe and a zwitterionic detergent. In this embodiment, the oligonucleotide probe is operatively coupled to a detectable label.

The invention is also directed to methods of utilizing the compositions of the invention. Accordingly, the invention provides a method for increasing the efficiency of a polymerase and a biochemical reaction involving a polymerase. In one embodiment, the method involves forming a reaction mixture by mixing a target nucleic acid with at least one polymerase, at least one primer, at least one oligonucleotide probe, at least one detectable label, dNTPs, and at least one zwitterionic detergent. At least one detectable label is operatively coupled to at least one oligonucleotide probe. In another embodiment, the method is performed by forming a reaction mixture which includes a target nucleic acid, a polymerase, a primer, dNTPs and at least one zwitterionic detergent. In embodiments, the reaction mixture does not contain a detectable label. In still another embodiment, the invention is directed to forming a reaction mixture that includes a target nucleic acid, a purified polymerase, a primer, a detectable label, nucleoside-5′-triphosphates, and a zwitterionic detergent or non-detergent surfactant. In some embodiments, a combination of two or more zwitterionic detergents are utilized. Also, in some embodiments, the reaction mixture is subjected to thermal cycling.

In yet another aspect, the invention is directed to a method of preparing a storage composition. The storage composition is formed by mixing at least one polymerase and at least one zwitterionic detergent or non-detergent surfactant in a suitable buffer. A combination of two or more zwitterionic detergents may comprise this method. In addition, in embodiments, the storage buffer does not contain a detectable label.

In yet a further aspect, the invention is directed to a method for detecting a target nucleic acid. In one embodiment, the method includes forming a reaction mixture that includes one or more polymerases, primers, zwitterionic detergents, dNTPs and detectable labels; subjecting the reaction mixture to nucleic acid amplification reaction conditions, which amplifies the target; and detecting a signal generated from the detectable label(s). The signal generated from the detectable label is indicative of the presence and/or amount of the target in the sample.

In embodiments, the method includes forming a reaction mixture that includes a polymerase, primer, zwitterionic detergent, dNTPs, and an oligonucleotide probe operatively coupled to an interactive pair of labels; subjecting the reaction mixture to nucleic acid amplification reaction conditions, which amplifies the target; and detecting a signal generated from a member of the interactive pair of labels. The signal generated is indicative of the presence and/or amount of the target in the sample.

In another aspect, the invention provides a way of stabilizing, storing, and/or enhancing the activity of a polymerase before or during a mutagenesis procedure. In one embodiment, the invention provides a method to make mutations in a nucleic acid molecule with the addition of a zwitterionic detergent and/or non-detergent surfactant to the reaction.

In still another aspect, the invention is directed to kits containing the compositions of the invention. The kit format may comprise a package unit having one or more containers of the subject composition, and in some embodiments, may include containers of various reagents used for polynucleotide synthesis, including synthesis in PCR, sequencing, mutagenesis, and the like. Generally, the kit includes at least one polymerase and at least one zwitterionic detergent and/or non-detergent surfactant. The kit may be used for increased stability during storage of a polymerase and/or for enhanced activity during the methods of the invention.

Any of the above aspects may be used with a non-detergent surfactant in place of the zwitterionic detergent, or a mixture of surfactant(s) and zwitterionic detergent(s). Suitable non-detergent surfactants are described herein and known in the art, including, but not necessarily limited to, the Air Products series of Surfynol surfactants (Surfynol 104, Surfynol 420, Surfynol440, Surfynol 465, Surfynol 485, Surfynol 504, Surfynol PSA series, Surfynol SE series, Dynol 604, Surfynol DF series, Surfynol CT series, and Surfynol EP series, for example Surfynol 104 series (104,104A, 104BC,104DPM, 104E, 104H, 104NP, 104PA, 104PG50, 1045), and Surfynol 2502). Although reference may be made herein to only a zwitterionic detergent, it is understood that a suitable non-detergent surfactant can be used as well. Likewise, it should be understood that reference to “a” detergent or surfactant includes reference to two or more.

### BRIEF DESCRIPTION OF THE DRAWINGS

FIG. 1 illustrates that individual zwitterionic detergents enhance the activity of Pfu fusion DNA polymerases in PCR reactions (Panels A, B and C).

FIG. 2 illustrates that combinations of zwitterionic detergents enhance activity of Pfu fusion DNA polymerases (Panels A, B, and C).

FIG. 3 further illustrates that combinations of zwitterionic detergents enhance activity of Pfu fusion DNA polymerases (Panels A, B and C).

FIG. 4 illustrates that zwitterionic detergents enhance the storage stability of Pfu DNA polymerases (Panels A and B).

FIG. 5 illustrates that individual zwitterionic detergents enhance the activity of Pfu DNA polymerases (Panels A, B and C).

FIG. 6 illustrates that zwitterionic detergents stabilize Pfu fusion DNA polymerases in accelerated stability studies (Panels A, B, C, D, E and F).

FIG. 7 illustrates that zwitterionic detergents enhance QPCR amplification with Pfu fusion DNA polymerases (Panels A, B, C and D).

FIG. 8 further illustrates that zwitterionic detergents enhance QPCR amplification with Pfu fusion DNA polymerases (Panels A, B, C and D).

FIG. 9 illustrates that Surfynol 465 enhances the activity of Pfu DNA polymerase (Panels A and B).

### DETAILED DESCRIPTION OF VARIOUS EMBODIMENTS OF THE INVENTION

The invention provides compositions, kits and methods that include a polymerase and a zwitterionic detergent or non-detergent surfactant. Such compositions and methods are useful in, among other things, the storage and use of DNA polymerases in thermal cycling reactions, including, but not limited to PCR and all of its variants (e.g., real-time PCR or quantitative PCR). The present invention is based at least in part on the surprising finding that zwitterionic detergents and non-detergent surfactants increase stability and enhance activity of thermostable DNA polymerases. For example, product yields are dramatically higher when PCR amplification reactions are conducted in buffers containing one or more zwitterionic detergents (e.g., CHAPS, CHAPSO, Anzergent 3-10, and Anzergent 3-12) or non-detergent surfactants (e.g., Surfynol 465). Similarly, zwitterionic detergents and non-detergent surfactants produce higher amplification efficiencies, higher total fluorescence, and earlier Ct values in QPCR reactions employing thermostable DNA polymerase and SYBR Green to monitor duplex DNA formation.

In general, the invention is directed to storage and reaction compositions having a polymerase and at least one zwitterionic detergent or non-detergent surfactant. In one embodiment, the storage and reaction compositions comprise a polymerase and both a zwitterionic detergent and non-detergent surfactant. Generally, a reaction mixture will include some or all of the necessary components to perform a nucleic acid synthesis reaction. A storage mixture may or may not include all the components necessary to perform a nucleic acid synthesis reaction.

The polymerases may be stored in a storage buffer comprising a zwitterionic detergent, a non-detergent surfactant, or both. The polymerases of the invention, described herein below, may be obtained commercially or produced by methods well known to one of skill in the art. The storage buffer and reaction buffers may include from about 0.001% to 5% volume/volume of each zwitterionic detergent or non-detergent surfactant employed.

As used herein, “zwitterionic detergent” or “zwitterionic surfactant” refers to detergents exhibiting zwitterionic character (e.g., does not possess a net charge, lacks conductivity and electrophoretic mobility, does not bind ion-exchange resins, breaks protein-protein interactions). Such compounds include, but are not limited to, CHAPS and sulfobetaines sold under the brand names Zwittergent® (Calbiochem, San Diego, Calif.) and Anzergent® (Anatrace, Inc., Maumee, Ohio). Particularly suitable detergents are known in the art and/or described below.

Generally the zwitterionic detergent will have the general formula:

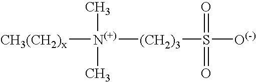

Zwitterionic detergents for use in practicing the invention include those sold under the brand names Zwittergent® and Anzergent®, having the chemical names of: n-Tetradecyl-N, N-dimethyl-3-ammonio-1-propanesulfonate, n-Octyl-N,N-dimethyl-3-ammonio-1-propanesulfonate, n-Decyl-N,N-dimethyl-3-ammonio-1-propanesulfonate, and n- Dodecyl-N,N-dimethyl-3-ammonio-1-propanesulfonate. Detergents of the present invention can be purchased under the brand names, for example, of Anzergent 3-14, Analytical Grade; Anzergent 3-8, Analytical Grade; Anzergent 3-10, Analytical Grade; Anzergent 3-12, Analytical Grade, or zwittergent 3-8, zwittergent 3-10, zwittergent 3-12 and zwittergent 3-14, CHAPS, CHAPSO, Apo10 and Apo12. Preferred zwitterionic detergents for practicing the invention include CHAPS, CHAPSO, Anzergent 3-10 and Anzergent 3-12.

As used herein, “non-detergent surfactant” refers to a composition that lowers surface tension and helps wet out surfaces, but does not have cleaning power (detergency). A “detergent” possesses cleaning power by sequestering dirt and oil in the interior of micelles formed by orienting detergent molecules with relatively small hydrophilic head groups toward the hydrophilic solvent (usually water) and hydrophobic tails (many carbon-carbon bonds, either straight chain alkyl or cyclic and/or polycyclic) toward the hydrophobic micelle interior. A non-detergent surfactant, in contrast, is a molecule with a relatively small hydrophobic head and two long hydrophilic ethylene oxide tails. The non-detergent surfactants lower surface tension but do not allow for micelle formation and detergency.

Non-detergent surfactants for use in practicing the invention include, but not necessarily limited to, those sold under the brand names Surfynol 104, Surfynol 420, Surfynol 440, Surfynol 465, Surfynol 485, Surfynol 504, Surfynol PSA series, Surfynol SE series, Dynol 604, Surfynol DF series, Surfynol CT series, and Surfynol EP series, for example Surfynol 104 series (104,104A, 104BC, 104DPM, 104E, 104H, 104NP, 104PA, 104PG50, 104S), and Surfynol 2502. Non-detergent surfactants are readily available from commercial suppliers.

Non-detergent surfactants for use in practicing the invention include, but not necessarily limited to, the DOWFAX series of Nonionic surfactants that are produced by polymerizing ethylene oxide (EO), propylene oxide (PO), and/or butylene oxide (BO) in the same molecule. These include those sold under the brand name, DOWFAX 63N10, DOWFAX 63N13, DOWFAX 63N30, DOWFAX 63N40, DOWFAX 81N13, DOWFAX 81N15, DOWFAX 92N20, DOWFAX 100N15, DOWFAX EM-51, DOWFAX 20A42, DOWFAX 20A64, DOWFAX 20A612, DOWFAX 20B102, DOWFAX DF-101, DOWFAX DF-111, DOWFAX DF-112, DOWFAX DF-113, DOWFAX DF-114, DOWFAX DF-117, DOWFAX WP-310, DOWFAX 50C15, DOWFAX DF-121, DOWFAX DF-122, DOWFAX DF-133, DOWFAX DF-141, DOWFAX DF-142, DOWFAX DF-16 (DOW Chemical Company, Midland, Mich.).

Non-detergent surfactants for use in practicing the invention include, but are not necessarily limited to, the PLURONIC® block copolymer series of surfactants having the general structure (C₂H₄O)ₐ(C₃H₆O)_{b}(C₂H₄O)ₐH. These include those sold under the brand name, PLURONIC® block copolymer series of surfactants (L35, P65, P75, P85, P103, P104, P105, F108) (BASF Corporation; Mount Olive, N.J.).

Other non-detergent surfactants include: Dimethylethylammonium-1-propanesulfonate, 3 -(1 -Pyridino)-1-propanesulfonate, Dimethyl-2-hydroxyethyl-1-propanesulfonate, 3-(1-Methylpiperidinium)-1-propanesulfonate, N-Methyl-N-(3-sulfopropyl)morpholinium and Dimethylbenzylammonium-l-propanesulfonate.

The zwitterionic detergent or non-detergent surfactant is used in an amount effective to induce the desired result (e.g., stabilize and/or enhance activity of a thermostable DNA polymerase). The optimal concentration of zwitterionic detergent or non-detergent surfactant for use in the compositions and methods will often vary between polymerases. One of skill in the art may perform routine testing to determine the optimal concentration of zwitterionic detergent or non-detergent surfactant for use with the particular polymerase. For example, a series of PCR reactions can be performed in which only the concentration of the detergent is varied (e.g., 0.05% to 1% Anzergent 3-10). The polymerase activity can then be determined by detecting and/or quantifying the amplified product by methods known in the art and described herein, (e.g., quantification by real-time PCR or gel electrophoresis of amplified product; see Examples 1-5). The most effective concentration of the zwitterionic detergent or non-detergent surfactant for use with the polymerase is the concentration which results in the most amplified product.

Similarly, test zwitterionic or non-detergent surfactants can be assayed for their effectiveness in amplification reactions by performing the assay described above and comparing the amount of amplified product produced in the composition comprising the test zwitterionic detergent or non-detergent surfactant to a negative control that does not include any surfactant.

The effectiveness of zwitterionic and non-detergent surfactants in stabilizing polymerases in a storage compositions can be assayed by similar methods. For example, the storage stability studies may be performed by storing the polymerase with the zwitterionic or non-detergent surfactant for a period of time (e.g., 1 week) at −20° C. The polymerase is then assayed for its ability to amplify a target nucleic acid as described above. Alternatively, an accelerated stability test may be performed in which the polymerase and zwitterionic detergent and/or non-detergent surfactant to be tested are subjected to 95° C. for 6 hours. The polymerase is then assayed for its ability to amplify a target nucleic acid and a comparison is made of the amount of amplified product in the reaction utilizing the zwitterionic and/or non-detergent surfactant to a reaction mixture that is surfactant free. If the amplified product is greater with the addition of the surfactant(s), then the tested surfactant(s) is effective at stabilizing the polymerase in a storage composition (see, for example, Example 3).

In one embodiment, the zwitterionic detergent is CHAPS. In certain embodiments, CHAPS is present at a concentration of about 0.05% to 1.0% volume/volume of the total composition. In other embodiments, CHAPS is present at a concentration of about 0.2% to 0.8% volume/volume of the total composition. In yet other embodiments, CHAPS is present at a concentration of about 0.2% to 0.4% volume/volume of the total composition.

In another embodiment, CHAPSO is present at a concentration of about 0.05% to 1.0% volume/volume of the total composition. In yet another embodiment, CHAPSO is present at a concentration of about 0.1 % to 0.4% volume/volume of the total composition. In a further embodiment, CHAPSO is present at a concentration of about 0. 15% to 0.35% volume/volume of the total composition.

In yet another embodiment, Anzergent 3-10 is present at a concentration of about 0.1% to 1.0% volume/volume ofthe total composition. In a further embodiment, Anzergent 3-10 is present at a concentration of about 0.4% to 0.8% volume/volume of the total composition.

In still another embodiment, Anzergent 3-12 is present at a concentration of about 0.05% to 1.0% volume/volume of the total composition. In still another embodiment, Anzergent 3-12 is present at a concentration of about 0.1 % to 0.4% volume/volume of the total composition. In a further embodiment, Anzergent 3-12 is present at a concentration of about 0.1% to 0.2% volume/volume of the total composition.

It is also envisioned that compatible zwitterionic detergents for use in the present invention can be mixed together to provide the requisite detergent for use in the invention. Generally, any two different zwitterionic or non-detergent surfactants may be present in a ratio of from 1:100 to 100:1, such as from 1:1, 1:2, 1:5, 1:10, 1:100, 100:1, 10:1, 5:1, or2:1. For example, the composition may include a combination of CHAPS and Anzergent 3-12; CHAPS and Anzergent 3-10; CHAPSO and Anzergent 3-12; or CHAPSO and Anzergent 3-10.

In one embodiment, CHAPS is present at a concentration of about 0.1% and Anzergent 3-12 is present at a concentration of 0.1% to 0.5%. In yet another embodiment, CHAPSO is present at a concentration of 0.1% and Anzergent 3-10 is present at a concentration of 0.05% to 0.5%. In a further embodiment, CHAPSO is present at a concentration of 0.1% and Anzergent 3-10 is present at a concentration of 0.05% to 0.4%. In yet another embodiment, CHAPSO is present at a concentration of 0.05% to 0.1% and Anzergent 3-12 is present at a concentration of 0.05% to 0.5%. In a further embodiment, CHAPSO is present at a concentration of 0.05% to 0.1% and Anzergent is present at a concentration of 0.05% to 0.4%. Additional zwitterionic detergent concentrations are illustrated in the Examples.

As mentioned above, one aspect of the invention relates to storage compositions. In one embodiment of this aspect, the storage composition comprises a polymerase and at least one zwitterionic detergent or non-detergent surfactant. The invention may provide a storage composition that includes a polymerase and a combination of two or more zwitterionic detergents or non-detergent surfactants. In certain embodiments, the storage composition does not contain a detectable label.

In one embodiment, the storage buffer comprises Tris-HCl or Tris-SO₄, and a pH of about 8-10. In a further embodiment, the storage buffer includes 50% (v/v) glycerol, 50 mM Tris-HCl (pH 8.2), 0.1 mM ethylenediaminetetraacetic acid (EDTA), and 1 mM dithiothreitol (DTT).

In another embodiment, the storage buffer includes 20 mM Tris-HCl (pH 8.8), 10 mM KCl, 10 mM (NH₄)₂SO₄, 2 mM MgSO₄, and 100 ug/ml BSA. In yet another embodiment, the storage buffer includes 40 mM Tris-SO₄ (pH 10), 15 mM K₂SO₄, 8 mM (NH₄)₂SO₄, and 2 mM MgSO₄. In still another embodiment, the storage buffer includes 30 mM Tris-SO₄ (pH 10), 40 mM K₂SO₄, 1.5 mM (NH₄)₂SO₄, and 2 mM MgSO₄. Other suitable storage buffers that are contemplated for use in the present invention and are known in the art.

In an embodiment, the invention is directed to a storage composition that includes a purified polymerase, a labeled nucleotide, and at least one zwitterionic detergent or non-detergent surfactant. In one embodiment, the labeled nucleotide has a single detectable label. For example, the single detectable label may be a fluorophore. In another embodiment, the labeled nucleotide has an interactive pair of labels. Suitable interactive pair of labels include a quencher and a fluorophore.

In another embodiment, the invention is directed to a storage composition that includes a purified polymerase, a fluorescent DNA binding dye, and a zwitterionic detergent or non-detergent surfactant, wherein said fluorescent DNA binding dye produces a detectable signal when bound to DNA. Suitable DNA binding dyes are known in the art and described herein. For example, DNA binding dyes include, but are not limited to, SYBR Green or EvaGreen.

It is contemplated that compositions of the invention will often include detectable labels. The detectable labels may be operatively coupled to the probe (e.g., FAM and BHQ2), may be provided free in solution (e.g., fluorescent DNA binding dyes, SYBR green), or operatively coupled to a nucleotide precursor.

The use of labeled probes in the amplification and quantification of a target polynucleotide (e.g., PCR) is described in many references, such as Innis et al., editors, “PCR Protocols” (Academic Press, New York, 1989); Sambrook et al., “Molecular Cloning”, Second Edition (Cold Spring Harbor Laboratory, New York, 1989), which are hereby incorporated herein by reference.

As used herein, the term “probe” or “oligonucleotide probe” refers to a single-stranded oligonucleotide having a sequence partly or completely complementary to a nucleic acid sequence sought to be detected, so as to stably hybridize thereto under stringent hybridization conditions. Probes may, but need not, have regions which are not complementary to a target sequence, as long as such sequences do not substantially alter the probe's desired specificity under stringent hybridization conditions.

In some embodiments, the probe is operatively coupled to a “label”. As used herein, the term “label” refers to any substance that can be used to provide a detectable (preferably quantifiable) signal, and which can be operatively linked to a nucleic acid. Labels may provide signals detectable by any suitable means, such as fluorescence, radioactivity, colorimetry, gravimetry, X-ray diffraction or absorption, magnetism, enzymatic activity, mass spectrometry, binding affinity, hybridization radio frequency, and the like.

In some embodiments, the probe is operatively coupled to an interactive pair of labels. As used herein, the phrase “interactive pair of labels” as well as the phrase “pair of interactive labels” as well as the phrase “first member and second member” refer to a pair of molecules which interact physically, optically, or otherwise in such a manner as to permit detection of their proximity by means of a detectable signal. Examples of a “pair of interactive labels” include, but are not limited to, labels suitable for use in fluorescence resonance energy transfer (FRET) (see, for example, Stryer, L. Ann. Rev. Biochem. 47, 819-846, 1978), scintillation proximity assays (SPA) (see, for example, Hart and Greenwald, Molecular Immunology 16:265267, 1979; U.S. Pat. No. 4,658,649), luminescence resonance energy transfer (LRET) (see, for example, Mathis, G. Clin. Chem. 41, 1391-1397,1995), direct quenching (see, for example, Tyagi et al., Nature Biotechnology 16, 49-53, 1998), chemiluminescence energy transfer (CRET) (see, for example, Campbell, A. K., and Patel, A. Biochem. J. 216, 185-194, 1983), bioluminescence resonance energy transfer (BRET) (see, for example, Xu, Y., Piston D. W., Johnson, Proc. Natl. Acad. Sc., 96, 151-156, 1999), or excimer formation (see, for example, Lakowicz, J. R. Principles of Fluorescence Spectroscopy, Kluwer Academic/Plenum Press, New York, 1999). The pair of labels can be either covalently or non-covalently attached to the oligonucleotide probes of the invention.

A pair of interactive labels useful for the invention can comprise a pair of FRET-compatible detectable labels, or a quencher-detectable label pair. In one embodiment, the pair comprises a fluorophore-quencher pair.

A wide variety of fluorophores can be used, including but not limited to: 5- FAM (also called 5-carboxyfluorescein; also called Spiro(isobenzofuran-1(3H), 9′-(9H)xanthene) -5-carboxylic acid, 3′,6′-dihydroxy-3-oxo-6-carboxyfluorescein); 5-Hexachloro -Fluorescein ([4,7,2′,4′,5′,7′-hexachloro-(3′,6′-dipivaloylfluoresceinyl)-6-carboxylic acid]); 6-Hexachloro-Fluorescein ([4,7,2′,4′,5′,7′-hexachloro-(3′,6′-dipivaloylfluoresceinyl)-5-carboxylic acid]); 5-Tetrachloro-Fluorescein ([4,7,2′,7′-tetra-chloro-(3′,6′-dipivaloylfluoresceinyl)-5-carboxylic acid]); 6-Tetrachloro-Fluorescein ([4,7,2′,7′-tetrachloro-(3′,6′-dipivaloylfluoresceinyl)-6-carboxylic acid]); 5-TAMRA (5-carboxytetramethylrhodamine; Xanthylium, 9-(2,4-dicarboxyphenyl)-3,6-bis(dimethyl-amino); 6-TAMRA (6-carboxytetramethylrhodamine; Xanthylium, 9-(2,5-dicarboxyphenyl)-3,6-bis(dimethylamino); EDANS (5-((2-aminoethyl)amino)naphthalene-1-sulfonic acid); 1,5-IAEDANS (5-((((2-iodoacetyl)amino)ethyl)amino)naphthalene-1-sulfonic acid); DABCYL (4-((4-(dimethylamino)phenyl)azo)benzoic acid) Cy5 (Indodicarbocyanine-5) Cy3 (Indo-dicarbocyanine-3); and BODIPY FL (2,6-dibromo-4,4-difluoro-5,7-dimethyl-4-bora-3a,4a-diaza-s-indacene-3-proprionic acid), Quasar-670 (Biasearch Technologies), CalOrange (Biosearch Technologies), Rox, as well as suitable derivatives thereof

As used herein, the term “quencher” refers to a chromophoric molecule or part of a compound, which is capable of reducing the emission from a fluorescent donor when attached to or in proximity to the donor. Quenching may occur by any of several mechanisms, including but not necessarily limited to fluorescence resonance energy transfer, photo-induced electron transfer, paramagnetic enhancement of intersystem crossing, Dexter exchange coupling, and exciton coupling such as the formation of dark complexes. Fluorescence is “quenched” when the fluorescence emitted by the fluorophore is reduced as compared with the fluorescence in the absence of the quencher by at least 10%, for example, 15%, 20%, 30%, 40%, 50%, 60%, 70%, 80%, 90%, 95%, 98%, 99%, 99.9% or more.

The quencher can be any material that can quench at least one fluorescence emission from an excited fluorophore being used in the assay. There is a great deal of practical guidance available in the literature for selecting appropriate reporter-quencher pairs for particular probes, as exemplified by the following references: Clegg (1993, Proc. Natl. Acad.Sci., 90:2994-2998); Wu et al. (1994, Anal. Biochem., 218:1-13); Pesce et al., editors, Fluorescence Spectroscopy (1971, Marcel Dekker, New York); White et al., Fluorescence Analysis: A Practical Approach (1970, Marcel Dekker, New York); and the like. The literature also includes references providing exhaustive lists of fluorescent and chromogenic molecules and their relevant optical properties for choosing reporter-quencher pairs, e.g., Berlman, Handbook of Fluorescence Spectra of Aromatic Molecules, 2nd Edition (1971, Academic Press, New York); Griffiths, Colour and Constitution of Organic Molecules (1976, Academic Press, New York); Bishop, editor, Indicators (1972, Pergamon Press, Oxford); Haugland, Handbook of Fluorescent Probes and Research Chemicals (1992 Molecular Probes, Eugene) Pringsheim, Fluorescence and Phosphorescence (1949, Interscience Publishers, New York), all of which incorporated hereby by reference. Further, there is extensive guidance in the literature for derivatizing reporter and quencher molecules for covalent attachment via common reactive groups that can be added to an oligonucleotide, as exemplified by the following references, see, for example, Haugland (cited above); Ullman et al., U.S. Pat. No. 3,996,345; Khanna et al., U.S. Pat. No. 4,351,760, all of which hereby incorporated by reference.

A number of commercially available quenchers are known in the art, and include but are not limited to DABCYL, BHQ-1, BHQ-2, and BHQ-3. The BHQ (“Black Hole Quenchers”) quenchers are a new class of dark quenchers that prevent fluorescence until a hybridization event occurs. In addition, these new quenchers have no native fluorescence, virtually eliminating background problems seen with other quenchers. BHQ quenchers can be used to quench almost all reporter detectable labels and are commercially available, for example, from Biosearch Technologies, Inc (Novato, Calif.).

Appropriate linking methodologies for attachment of many detectable labels to oligonucleotides are described in many references, e.g., Marshall, Histochemical J., 7: 299-303 (1975); Menchen et al., U.S. Pat. No. 5,188,934; Menchen et al., European Patent Application 87310256.0; and Bergot et al., International Application PCT/US90/05565. All are hereby incorporated by reference.

In some embodiments, the compositions of the invention include a detectable label. The detectable label may be any detectable label which will produce a signal indicative of the presence or amount of a target nucleic acid. Such detectable labels are known in the art and described above. Detectable labels useful according to the invention include fluorescent detectable labels such as SYBR green and FAM. In one embodiment, the label does not convert electromagnetic energy into thermal energy in order to heat the reaction mixture (e.g., as described in U.S. Patent Application Publication No.: US 2003/0017567, which is herein incorporated by reference in its entirety).

In one embodiment, the label is operatively coupled to a nucleotide. In one embodiment, the labeled nucleotide is a dual labeled nucleotides, as described in U.S. Patent Application Publication No. 2004/0014096, which is herein incorporated by reference in its entirety. The dual labeled nucleotide includes a fluorescent label and a quencher of that fluorescent label.

Other suitable dual labeled nucleotides include, for example, those taught in Rosenblum et al. (1997, Nucleic Acids Research, 25: 4500). Rosenblum et al. teaches the use of nucleotide analogs comprising a fluorescence resonance energy transfer (FRET) dye pair linked to the nucleobase. Incorporation of such analogs into a growing polynucleotide chain is detected by contacting the analog with light of a wavelength within the excitation spectrum of one of the dyes but not the other. The light emitted by the excited fluorophore then, in turn, excites the second dye, from which fluorescence emission is detected. In addition, Williams (U.S. Patent Application Publication No. 2001/0018184) teaches a dual-labeled nucleotide analog in which a fluorophore is attached to the gamma-phosphate of the polyphosphate moiety, and a quencher is linked elsewhere on the nucleotide analog, preferably linked to the 5′ carbon of pyrimidine bases and to the 7′ carbon of deazapurine bases. Upon incorporation of the analog taught by Williams into a growing polynucleotide chain, the phosphate group linked to the fluorescent moiety is cleaved off, thus separating the fluorescent moiety and the quencher, thereby permitting the fluorescent moiety to emit a detectable signal.

As discussed above, in another general aspect, the invention provides reaction mixtures. In one embodiment, the invention is directed to a reaction mixture that includes a polymerase and at least one zwitterionic detergent and/or non-detergent surfactant. The reaction buffer is useful for the amplification of a target nucleic acid, among other things. The reaction buffer comprises from about 0.001% to about 5% volume/volume of each zwitterionic detergent or non-detergent surfactant employed. In another embodiment, the invention provides a reaction mixture that includes a polymerase, an oligonucleotide probe, and at least one zwitterionic detergent and/or non-detergent surfactant. In embodiments, a detectable label is operatively coupled to the oligonucleotide probe. In yet other embodiments, the invention provides a reaction mixture that includes a polymerase, a detectable label, and at least one zwitterionic detergent or non-detergent surfactant. A combination of two or more zwitterionic detergents or non-detergent surfactants or a combination thereof can comprise the reaction mixture. The detectable label can, in some situations, be operatively coupled to the oligonucleotide probe. In other situations, the detectable label can comprise an interactive pair of labels.

In another embodiment of the invention directed to reaction mixtures, the invention is a mixture that comprises a composition having a purified polymerase, a labeled nucleotide, and at least one zwitterionic detergent or non-detergent surfactant. In one example, the labeled nucleotide has a single detectable label. For example, the single detectable label may be a fluorophore. In another example, the labeled nucleotide has an interactive pair of labels. A suitable interactive pair of labels includes a quencher and a fluorophore.

The reaction mixture can include a purified polymerase, a fluorescent DNA binding dye, and at least one zwitterionic detergent or non-detergent surfactant, where the fluorescent DNA binding dye produces a detectable signal when bound to DNA. Suitable DNA binding dyes are known in the art and described herein. For example, DNA binding dyes include, but are not limited to, SYBR Green or EvaGreen.

Where desired, the composition can be a reaction mixture that includes nucleoside-5′-triphosphates, primers, a buffer in which primer extension can occur, a polymerase, an oligonucleotide probe, and at least one zwitterionic detergent. In one example, the oligonucleotide probe is operatively coupled to a detectable label. In another example, the detectable label comprises an interactive pair of labels.

One non-limiting example of a reaction mixture is one that comprises a buffered composition having Tris-HCl or Tris-SO₄ (to achieve a final pH of about 8.0 to about 10), KCl or K₂SO₄, (NH₄)₂SO₄ and MgSO₄- In another non-limiting example, the reaction mixture comprises a buffered composition that includes Tris-HCl (pH 8.8), KCl, (NH₄)₂SO₄ and MgSO₄. In yet another non-limiting example, the reaction mixture comprises a buffered composition that includes 20 mM Tris-HCl (pH 8.8), 10 mM KCl, 10 mM (NH₄)₂SO₄, 2 mM MgSO₄, and 100 ug/ml BSA. In yet a further non-limiting example, the reaction buffer includes 40 mM Tris-SO₄ (pH 10), 15 mM K₂SO₄, 8 mM (NH₄)₂SO₄, and 2 mM MgSO₄. In still another non-limiting example, the reaction buffer includes 30 mM Tris-SO₄ (pH 10), 40 mM K₂SO₄, 1.5 mM (NH₄)₂SO₄, and 2 mM MgSO₄.

Additional storage and reaction buffers useful in practicing the invention are known in the art (e.g., those described in U.S. Patent Application Publication No. 2005/0048530; and U.S. patent application Ser. No. 11/152,773, filed Jun. 15, 2006, each of which is herein incorporated by reference in its entirety) and described in the Examples. For example, a composition may include a thermostable DNA polymerase, the buffer described in U.S. patent application Ser. No. 11/152,773, which comprises tris(2carboxyethyl)phosphine (TCEP) or similar phosphine compounds, and a non-ionic surfactant. In this embodiment, the non-ionic surfactant is a non-detergent non-ionic surfactant such as the Surfynol series of surfactants.

In another embodiment, the composition includes a thermostable polymerase, a zwitterionic or non-detergent surfactant (e.g., Surfynol series) and a buffer comprising potassium sulfate and ammonium sulfate which has a potassium sulfate:ammonium sulfate molar ratio of 5:1 to 50:1. In some embodiments, the potassium sulfate concentration ranges from 20 mM to 50 mM, and the ammonium sulfate concentration ranges from 1 to 5 mM.

The buffer for use in the compositions and methods of the invention are suitable for a variety of polymerases, and will be tailored for a particular polymerase. Suitable buffers are known in the art and described in the literature provided by the commercial source of the polymerase.

Any of the above listed aspects may be performed with a non-detergent surfactant or zwitterionic detergent. Suitable non-detergent surfactants include the Air Products series of Surfynol surfactants, including, but not necessarily limited to, Surfynol 104, Surfynol 420, Surfynol 440, Surfynol 465, Surfynol 485, Surfynol 504, Surfynol PSA series, Surfynol SE series, Dynol 604, Surfynol DF series, Surfynol CT series, and Surfynol EP series, Surfynol 104 series (104, 104A, 104BC, 104DPM, 104E, 104H, 104NP, 104PA, 104PG50, 104S), and Surfynol 2502, for example. Non-detergent surfactants are readily available from commercial suppliers.

The zwitterionic detergent/non-detergent surfactant is used in combination with the nucleic acid polymerase. As used herein, “nucleic acid polymerase” or “polymerase” refers to an enzyme that catalyzes the polymerization of nucleotides. Generally, the enzyme will initiate synthesis at the 3′-end of the primer annealed to a nucleic acid template sequence, and will proceed in the 5′-direction along the template. “DNA polymerase” catalyzes the polymerization of deoxynucleotides. Known DNA polymerases include, for example, Pyrococcus furiosus (Pfu) DNA polymerase, E. coli DNA polymerase I, T7 DNA polymerase, Thermus thermophilus (Tth) DNA polymerase, Bacillus stearothermophilus DNA polymerase, Thermococcus litoralis (Tli) DNA polymerase (also referred to as Vent DNA polymerase), Thermotoga maritima (UlTma) DNA polymerase, Thermus aquaticus (Taq) DNA polymerase, and Pyrococcus GB-D(PGB-D) DNA polymerase. DNA polymerases and their properties are described in detail in, among other places, DNA Replication 2nd edition, Kornberg and Baker, W. H. Freeman, New York, N.Y. (1991). Known conventional DNA polymerases include, for example, Pyrococcus furiosus (Pfu) DNA polymerase (Lundberg et al., 1991, Gene 108:1, provided by Stratagene, La Jolla, Calif., USA), Pyrococcus woesei (Pwo) DNA polymerase (Hinnisdaels et al., 1996, Biotechniques 20:186-8), Thermus thermophilus (Tth) DNA polymerase (Myers and Gelfand 1991, Biochemistry 30:7661), Bacillus stearothermophilus DNA polymerase (Stenesh and McGowan, 1977, Biochim. Biophys. Acta 475:32), Thermococcus litoralis (Tli) DNA polymerase (also referred to as Vent DNA polymerase, Cariello et al., 1991, Polynucleotide Res. 19: 4193, available from, e.g., New England Biolabs, Beverly, Mass., USA), 9° Nm DNA polymerase, Thermotoga maritima (Tma) DNA polymerase (Diaz and Sabino, 1998. Braz. J. Med. Res. 31:1239), Thermus aquaticus (Taq) DNA polymerase (Chien et al., 1976, J Bacteriol. 127:1550), Pyrococcus kodakaraensis KOD DNA polymerase (Takagi et al., 1997, Appl. Environ. Microbiol. 63:4504), JDF-3 DNA polymerase (from Thermococcus sp. JDF-3, Published International patent application WO 01/32887), Pyrococcus GB-D (PGB-D) DNA polymerase (also referred as Deep-Vent DNA polymerase, Juncosa-Ginesta et al., 1994, Biotechniques 16:820, available from, e.g., New England Biolabs, Beverly, Mass., USA), UlTma DNA polymerase (from thermophile Thermotoga maritima; Diaz and Sabino, 1998, Braz. J. Med. Res. 31:1239; available from, e.g., PE Applied Biosystems, Foster City, Calif., USA), Tgo DNA polymerase (from Thermococcus gorgonarius, available from, e.g., Roche Molecular Biochemicals, Indianapolis, Ind., USA), E. coli DNA polymerase 1 (Lecomte and Doubleday, 1983, Polynucleotide Res. 11:7505), T7 DNA polymerase (Nordstrom et al., 1981, J. Biol. Chem. 256:3112), and archaeal DP1/DP2 DNA polymerase II {Cann et al., 1998, Proc. Natl. Acad. Sci. USA 95:14250-5).

While not required, preferably, the polymerase is a purified polymerase. As used herein, a “purified” or “isolated” substance is any substance that has been separated from at least one other substance found naturally associated with the substance. Thus, as “purified polymerase” refers to a polymerase that has been separated from one or more components that naturally accompany it. These components may include, but are not limited to, cell components, such as nucleic acids, lipids, carbohydrates, other proteins, and other cell components released upon lysis of a cell containing the polymerase. To be considered highly purified, the polymerase may be about 50% or more purified from other cell components. In some embodiments, it is at least 60%, 70%, 80%, 90%, or 99% or more purified. More than one type of purified polymerase may be used in the invention, and each can be of an independent level of purity.

The term, “nucleic acid polymerase” also encompasses reverse transcriptases including, but not limited to, reverse transcriptases from HIV, HTLV-1, HTLV-II, FeLV, FIV, SIV, AMV, MMTV, MoMuLV and other retroviruses (for reviews, see for example, Levin, 1997, Cell 88:5-8; Verma, 1977, Biochim. Biophys. Acta 473:1-38; Wu et al, 1975, CRC Crit. Rev. Biochem. 3:289-347).

When using the subject compositions in reaction mixtures that are exposed to elevated temperatures (e.g., during the PCR technique), use of thermostable DNA polymerases is preferred. As used herein, “thermostable” refers to a property of a nucleic acid polymerase, such that the enzyme is active at elevated temperatures and is resistant to nucleic acid duplex-denaturing temperatures in the range of about 93° C. to about 100° C. “Active” means the enzyme retains the ability to effect primer extension reactions when subjected to elevated or denaturing temperatures for the time necessary to effect denaturation of double-stranded nucleic acids. Elevated temperatures as used herein refer to the range of about 70° C. to about 100° C., whereas non-elevated temperatures as used herein refer to the range of about 35° C. to about 50° C.

Thermostable DNA polymerases that may be used in the invention include, but are not necessarily limited to, Taq, Tne, Tma, Pfu, Tfl, Tth, Stoffel fragment, VENT™ and DEEPVENT™ DNA polymerases, KOD, Tgo, JDF3, and mutants, variants and derivatives thereof (see, for example, U.S. Pat. No. 5,436,149; U.S. Pat. No. 4,889,818; U.S. Pat. No. 4,965,18S; U.S. Pat. No. 5,079,352; U.S. Pat. No. 5,614,365; U.S. Pat. No. 5,374,553; U.S. Pat. No. 5,270,179; U.S. Pat. No. 5,047,342; U.S. Pat. No. 5,512,462; WO 92/06188; WO 92/06200; WO 96/10640; Barnes, W. M., Gene 112:29-35 (1992); Lawyer, F. C., et al., PCR Meth. Appl. 2:275-287 (1993); and Flaman, J.-M, et al., Nuc. Acids Res. 22(15):3259- 3260 (1994)).

In one embodiment, the thermostable DNA polymerase is a Pfu DNA polymerase or a Taq DNA polymerase. In another embodiment, the thermostable DNA polymerase is Pfu DNA polymerase with a mutation at position V93, wherein the polymerase is exonuclease deficient (e.g., Pfu V93, exo-). Methods of making and using Pfu V93, exo- DNA polymerase are described in U.S. patent application Ser. No.: 10/298,680, filed Nov. 18, 2002 and incorporated herein by reference in its entirety. In another embodiment, the polymerase is a fusion protein having polymerase activity (e.g., Pfu DNA polymerase-Sso7, as described in U.S. patent application Ser. No.: 11/488,535, filed Jul. 17, 2006, and U.S. Patent Application Publication No. 2005/0048530, filed Mar. 14, 2004, both of which are herein incorporated by reference in their entirety).

The zwitterionic detergent/non-detergent surfactant and polymerase compositions described herein may be used in any application for which polymerases are known to be used (e.g., nucleic acid amplification, PCR, QPCR, sequencing mutagenesis). As used herein, the term “nucleic acid amplification” refers to the production of additional copies of a nucleic acid sequence and is generally carried out using polymerase chain reaction (PCR) or ligase chain reaction (LCR) technologies well known in the art (see, for example, Dieffenbach, C. W. and G. S. Dveksler (1995) PCR Primer, a Laboratory Manual, Cold Spring Harbor Press, Plainview, N.Y.).

For ease of understanding certain advantages provided by the present invention, a summary of PCR is provided. The PCR reaction involves a repetitive series of temperature cycles and is typically performed in a volume of 50-100 ul. The reaction mix comprises dNTPs (each of the four deoxynucleotides dATP, dCTP, dGTP, and dTTP), primers, buffers, DNA polymerase, and polynucleotide template. PCR requires two primers that hybridize with the double-stranded target polynucleotide sequence to be amplified. In PCR, this double-stranded target sequence is denatured and one primer is annealed to each strand of the denatured target. The primers anneal to the target polynucleotide at sites removed from one another and in orientations such that the extension product of one primer, when separated from its complement, can hybridize to the other primer. Once a given primer hybridizes to the target sequence, the primer is extended by the action of a DNA polymerase. The extension product is then denatured from the target sequence, and the process is repeated. In successive cycles of this process, the extension products produced in earlier cycles serve as templates for DNA synthesis. Beginning in the second cycle, the product of amplification begins to accumulate at a logarithmic rate. The amplification product is a discrete double-stranded DNA molecule comprising: a first strand which contains the sequence of the first primer, eventually followed by the sequence complementary to the second primer, and a second strand which is complementary to the first strand.

The invention provides a method for increasing the efficiency of a polymerase. In one embodiment, the method involves forming a reaction mixture by mixing a target nucleic acid with a polymerase, a primer, an oligonucleotide probe, a detectable label, dNTPs and at least one zwitterionic detergent or non-detergent surfactant. In this embodiment, a combination of two or more zwitterionic detergents or non-detergent surfactants can be utilized. In one example, the detectable label is operatively coupled to the oligonucleotide probe. In a further embodiment, the reaction mixture is subjected to thermal cycling, which comprises subjecting a reaction mixture to two or more different incubation temperatures for a period of time. In one example, the denaturing step of a nucleic acid amplification reaction is at 95° C. for 1 minute and the annealing/extension step is at 65° C. for 30 s. The increase in efficiency of the polymerase results in more amplification product at the end of the method.

In another embodiment, the invention provides a method for increasing the efficiency of a polymerase without the use of a detectable label. The method is performed by forming a reaction mixture which includes a target nucleic acid, a polymerase, a primer, dNTPs and at least one zwitterionic detergent or non-detergent surfactant. In this embodiment, the reaction mixture can comprise a combination of two or more zwitterionic detergents or non-detergent surfactants. In a further embodiment, the reaction mixture is subjected to thermal cycling.

In yet another embodiment, the invention is directed to a method of increasing the efficiency of a polymerase by forming a reaction mixture which includes a target nucleic acid, a purified polymerase, a primer, a detectable label, nucleoside-5′-triphosphates, and at least one zwitterionic detergent or non-detergent surfactant. In one example, the detectable label is a labeled nucleotide. In a further example, the labeled nucleotide has a single detectable label. For example, the single detectable label may be a fluorophore. In another example, the labeled nucleotide has an interactive pair of labels. A suitable interactive pair of labels includes a quencher and a fluorophore. In still another example, the detectable label is a fluorescent DNA binding dye, wherein the fluorescent DNA binding dye produces a detectable signal when bound to DNA. Suitable DNA binding dyes are known in the art and described herein. For example, DNA binding dyes include, but are not limited to, SYBR Green or EvaGreen.

In another general aspect of the invention, the invention is directed towards a method of preparing a storage composition. The method comprises combining (e.g., mixing) a polymerase and at least one zwitterionic detergent in a suitable buffer to form a storage composition. A combination of two or more zwitterionic detergents may be used in the method. In some embodiments, the storage composition does not contain a detectable label.

In yet another general aspect, the invention provides methods for detecting a target nucleic acid. In one embodiment, the method includes forming a reaction mixture that comprises a polymerase, primer, zwitterionic detergent or non-detergent surfactant, dNTPs and a detectable label; subjecting the reaction mixture to nucleic acid amplification reaction conditions, which amplify the target; and detecting a signal generated from the detectable label. The signal generated from the detectable label is indicative of the presence and/or amount of the target in the sample. The reaction mixture may further include an oligonucleotide probe. In addition, the oligonucleotide probe and detectable label may be operatively coupled. Also, the detectable label may be an intercalating detectable label (e.g., SYBR green).

In another embodiment, the invention provides another way to detect a target nucleic acid. The method includes forming a reaction mixture that comprises a polymerase, primer, zwitterionic detergent or non-detergent surfactant, dNTPs, and an oligonucleotide probe operatively coupled to an interactive pair of labels; subjecting the reaction mixture to nucleic acid amplification reaction conditions, which amplify the target; and detecting a signal generated from a member of the interactive pair of labels. The signal generated is indicative of the presence and/or amount of the target in the sample.

As used herein, “nucleic acid amplification reaction conditions” refer to a composition (typically a buffered composition) and a set of temperature incubation steps and times that are possible and preferably optimal for conducting amplification of a nucleic acid. Amplification means an increase in the number of a particular nucleic acid sequence and may be accomplished, without limitation, by the in vitro methods of PCR, ligase chain reaction, or any other method of amplification. Such reaction conditions are known in the art or are described herein. Nucleic acid reaction conditions encompass PCR reaction conditions. In one embodiment, the step of subjecting the reaction mixture to nucleic acid amplification reaction conditions includes the step of heating the reaction mixture with a thermal cycler sample block so as to denature the target nucleic acid.

In one embodiment, the oligonucleotide probe is cleaved by a 5′ nuclease during the amplification reaction. In yet a further embodiment, the probe is cleaved, thereby separating the members of the interactive pair of labels and generating a detectable signal. Such methods are known in the art and described in, for example, U.S. Pat. Nos.: 6,528,254; 6,548,250 and; 5,210,015, which are each herein incorporated by reference in their entirety.

In another aspect, the zwitterionic detergent or non-detergent surfactant is used in a mutagenesis reaction to modify a nucleic acid molecule. For example, a zwitterionic detergent or non-detergent surfactant may be used in place of Triton-X 100 in the QUICKCHANGE site directed mutagenesis kit (Stratagene catalog #200518). The detergent or surfactant may be added before the mutagenesis reaction takes place, as a means, for example, to stabilize the polymerase during storage, or may be added in the reaction to enhance activity of the polymerase. The method comprises contacting the polymerase with an amount of zwitterionic detergent and/or non-detergent surfactant that is effective in stabilizing the polymerase during storage and/or enhances the activity of the polymerase during the mutagenesis reaction.

As discussed above, the invention provides novel compositions and methods having at least one zwitterionic detergent and/or non-detergent surfactant and a polymerase. The invention further provides a kit that comprises a package unit having one or more containers of the composition, and in some embodiments, includes containers of various reagents used for polynucleotide synthesis, including synthesis in PCR, sequencing, mutagenesis, and the like. Among other things, the kit may also contain one or more of the following items: polynucleotide precursors (e.g., nucleoside triphosphates), primers, probes, buffers, instructions, labeled nucleotides, intercalating dyes, and control reagents. The kit may include containers of reagents mixed together in suitable proportions for performing the methods in accordance with the invention. Reagent containers preferably contain reagents in unit quantities that obviate measuring steps when performing the subject methods. One exemplary kit according to the invention also contains a DNA yield standard for the quantitation of the PCR product yields from a stained gel.

In one embodiment, the kit includes a master mix reagent comprising a thermostable polymerase, a zwitterionic or non-detergent surfactant, and polynucleotide precursors. In another embodiment, the kit includes a storage and/or reaction buffer having a polymerase and at least one zwitterionic detergent or non-detergent surfactant. The storage buffer does not contain a detectable label in some configurations. A combination of two or more zwitterionic detergents or non-detergent surfactants may be provided. In yet another embodiment, the kits may further include a separate container having dNTPs. In another embodiment, any of the above kits may further include a separate container having a detectable label.

In an embodiment, the invention is directed to a kit which includes a purified polymerase, at least one zwitterionic detergent or non-detergent surfactant, polynucleotide precursors, and a labeled nucleotide. In yet another embodiment, the invention is directed to a kit which includes a purified polymerase, a zwitterionic detergent or non-detergent surfactant, polynucleotide precursors, and a DNA binding dye.

In some embodiments of the kits, the zwitterionic detergent and/or non-detergent surfactant is provided as a concentrated stock for use after dilution. For example, it may be provided at a 10× concentration in a 10× stock reaction buffer that is suitable for performing a nucleic acid amplification reaction. The 10× stock is diluted to a final 1× working concentration.

### EXAMPLES

The invention will be further explained by the following Examples, which are intended to be purely exemplary of the invention, and should not be considered as limiting the invention in any way.

### Example 1

### Preparing Polymerases and Buffers Lacking Conventional Non-Ionic Detergents

Pfu (exo+ and exo−) fusion DNA polymerase (e.g., as described in U.S. patent application Ser. No.: 11/488,535, filed Jul. 17, 2006, and herein incorporated by reference in its entirety), cPfu DNA polymerase (Stratagene catalog #600154), and PEF were purified using standard production protocols (no detergent present), except that non-ionic detergents were omitted from the final storage buffers. Enzymes were stored at −20° C. in 50 mM Tris-HCl (pH 8.2), 0.1 mM EDTA, 1 mM DTT, and 50% glycerol. DNA polymerase storage buffers were additionally supplemented with one or more zwitterionic detergents, in percentages (v/v) ranging from 0.05% to 0.5%.

PCR reaction buffers were prepared without non-ionic detergents (“DF buffer”, detergent-free buffer). For example, 1× cPfu DF-buffer contains 10 mM KCl, 10 mM (NH₄)₂SO₄, 20 mM Tris HCl (pH 8.8), 2 mM MgSO₄, and 100 ug/ml BSA. Detergent-free versions of Pfu fusion buffers were also prepared, and consisted of: 40 mM Tris-SO₄ (pH 10), 15 mM K₂SO₄, 8 mM (NH₄)₂SO₄, 2 mM MgSO₄ (1× Pfu fusion DF-buffer I) or 30 mM Tris-SO₄ (pH 10), 40 mM K₂SO₄, 1.5 mM (NH₄)₂SO₄, 2 mM MgSO₄ (1× Pfu fusion DF-buffer II).

PCR reaction buffers were supplemented with 0.1% Triton X100 (non-ionic detergent) or with one or more zwitterionic detergents or non-detergent surfactants. For example, zwitterionic detergents CHAPS, CHAPSO, 3-10, and 3-12 were obtained from AnaTrace, Inc. (Maumee, Ohio) and added to DF-buffers in percentages (v/v) ranging from 0.05% to 0.5%. The non-detergent surfactant, Surfynol 465, was purchased from Air Products and used in a similar fashion.

### Example 2

### Using Zwitterionic Detergents to Enhance Pfu and Pfu Fusion DNA Polymerase Activity in Endpoint PCR

For the 0.9 kb and 6 kb systems, PCR reactions (50 ul) were conducted with 40 ng cPfu DNA polymerase in IX cPfu DF-buffer or with 28 ng or 224 ng Pfu fusion DNA polymerase in 1× Pfu fusion DF-buffer I or DF-buffer II, respectively. PCR reactions also contained 2 U/50 ul Pyrococcus furiosus dUTPase (PEF), 100 ng of human genomic DNA, 250 uM each dNTP, and 100 ng of each primer. For the 9 kb system, PCR reactions (50 ul) consisted of 80 ng Pfu, 1.5× cPfu DF-buffer, 2U Pyrococcus furiosus dUTPase (PEF), 200 ng of human genomic DNA, 500 uM each dNTP, and 200 ng of each primer. PCR reaction buffers were supplemented with 0.1% Triton X100 or with zwitterionic detergent(s). Reactions were cycled as described below:

TABLE 1

Reaction Conditions
Endpoint PCR Systems

| Target size | | |
|---|---|---|
| (gene) | Cycling parameters | Primer sequence |
| 0.9kb | Pfu fusion: (1 cycle) 95° C. 2 min; (30 | F-5′-AGA.GCT.TGA.GGA.GAG. |
| (Hα1AT) | cycles) 95° C. 20 sec, 58° C. 20 sec, 72° C. | CAG.GAA.AGG.TGG.AAC-3′ |
| | 15-30 sec.; (1 cycle) 72° C. 3 min | (SEQ ID NO: 1) |
| | Pfu: (1 cycle) 95° C. 2 min; (30 cycles) | R-5′-GGG.AGG.GGA.GGT.ACA. |
| | 95° C. 30 sec, 58° C. 30 sec, 72° C. 60 sec.; | GGG.TTG.AGG.CTA.GTG-3′ |
| | 1 cycle) 72° C. 10 min | (SEQ ID NO: 2) |
| 6 kb | Pfu fusion: (1 cycle) 95° C. 2 min; (30 | F-5′-ACA.AGG.GCT.ACT.GGT. |
| (β-globulin) | cycles) 95° C. 20 sec, 58° C. 20 sec, 72° C. | TGC.CGA.TTT.TTA.TTG-3′ |
| | 90-180 sec.; (1 cycle) 72° C. 3 min | (SEQ ID NO: 3) |
| | Pfu: (1 cycle) 95° C. 2 min; (30 cycles) | R-5′-GGG.ACT.GGC.CTC.AGA. |
| | 95° C. 30 sec, 58° C. 30 sec, 72° C. 12 | GGA.AAC.TTC.AGG-3′ |
| | min.; (1 cycle) 72°° C. 10 min | (SEQ ID NO: 4) |
| 9kb | Pfu: (1 cycle) 95° C. 2 min; (30 cycles) | F-5′-ACA.AGG.GCT.ACT.GGT. |
| (β-globulin) | 92° C. 30 sec, 58° C. 30 sec, 68° C. 18 | TGC.CGA.TTT.TTA.TTG-3′ |
| | min.; (1 cycle) 68° C. 10 min | (SEQ ID NO: 3) |
| | | R-5′-GTT.TGA.GCA.ACT.CTC. |
| | | ACC.ATT.ATG.GGC-3′ |
| | | (SEQ ID NO: 5) |

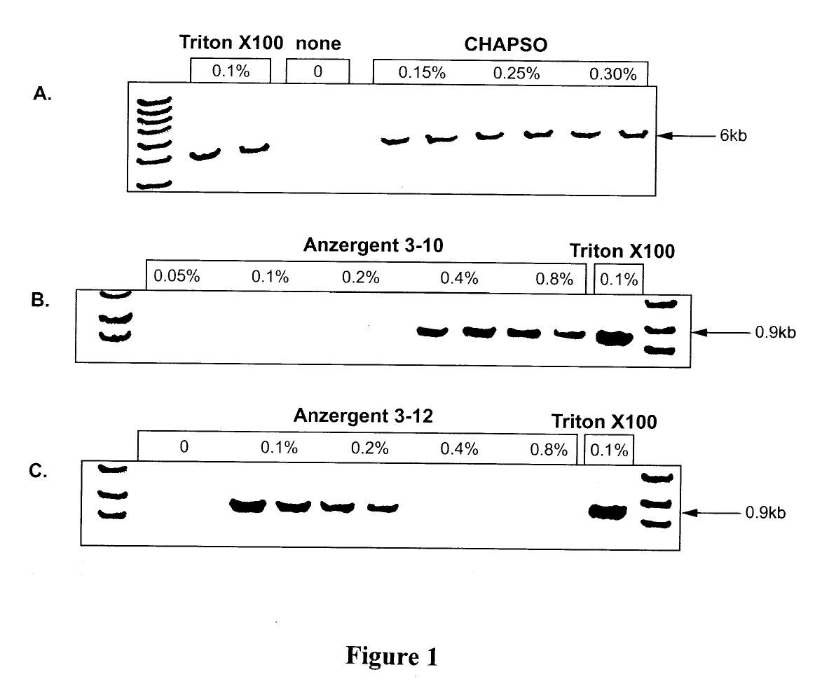

The results shown in FIGS. 1-8 demonstrate the enhancing and/or stabilizing activity of zwitterionic detergents. Pfu fusion DNA polymerase, purified and stored in the absence of detergent, was used to amplify genomic DNA targets in fusion PCR buffers supplemented with zwitterionic detergents (FIGS. 1-3). As shown in FIG. 1, PCRs conducted in the absence of detergent failed to generate product (Panel A, lanes 4 and 5). In contrast, amplifications performed in the presence of CHAPSO (0.15-0.3%; Panel A, lanes 6-11), Anzergent 3-10 (0.4-0.8%; Panel B, lanes 8-11), and Anzergent 3-12 (0.1-0.2%; Panel C, lanes 4-7) generated product yields.

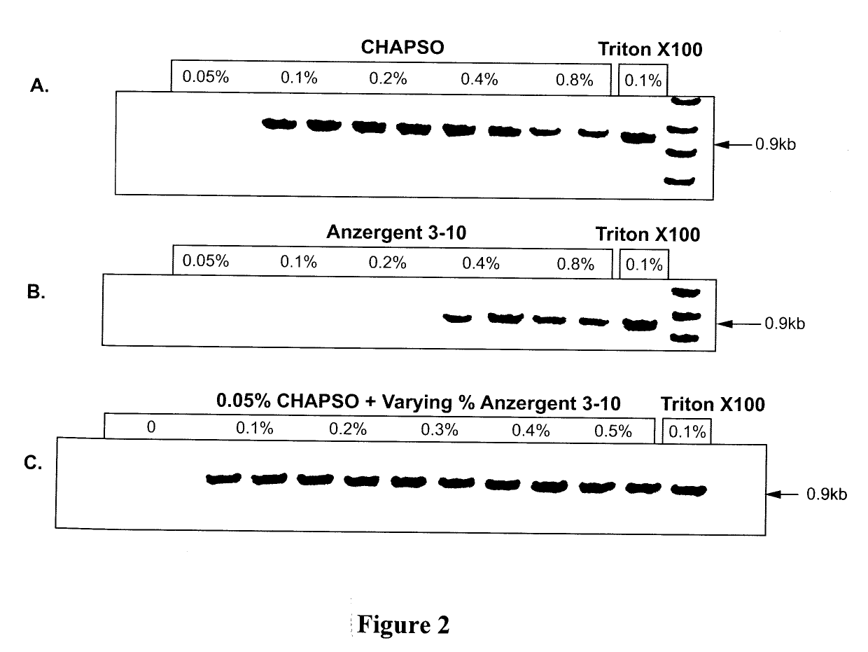

FIG. 2 depicts more amplification results when detergent is added to the reaction (0.05-0.8%; Panel A, lanes 2-11). As can be seen, the addition of 0.1% to 0.4% CHAPSO had a favorable result on PCR amplification. The addition of 0.4% and 0.8% Anzergent 3-10 also yielded favorable results (Panel B, lanes 8-11). The addition of 0.05% CHAPSO (Panel A, lanes 2-3) or 0.05% Anzergent 3-10 (Panel B, lanes 2-3) resulted in little amplification during the reaction. However, when suboptimal amounts of CHAPSO (0.05%) was combined with suboptimal amounts of Anzergent 3-10 (0.1%-0.2%), greater enhancement of Pfu fusion activity was seen ( Panel C, lanes 4-7). Therefore, in some cases, combinations of zwitterionic detergents provide greater enhancement of Pfu fusion activity than individual detergents.

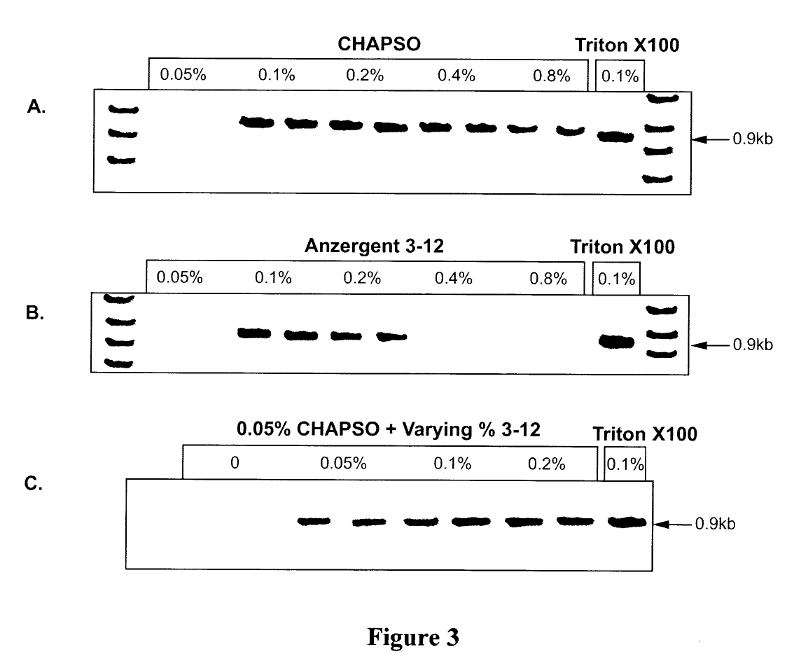

The same effect was seen in FIG. 3 when CHAPSO and Anzergent 3-12 were combined in an amplification assay. In this case, the addition of 0.05% CHAPSO or 0.05% Anzergent 3-12 by themselves to the reaction did not show observable levels of PCR product (Panel A, lanes 2-3 and Panel B, lanes 2-3, respectively). However, product yields were dramatically improved when suboptimal amounts of 3-12 (0.05%) were combined with a suboptimal amount of CHAPSO (0.05%) detergent (Panel C, lanes 4-5).

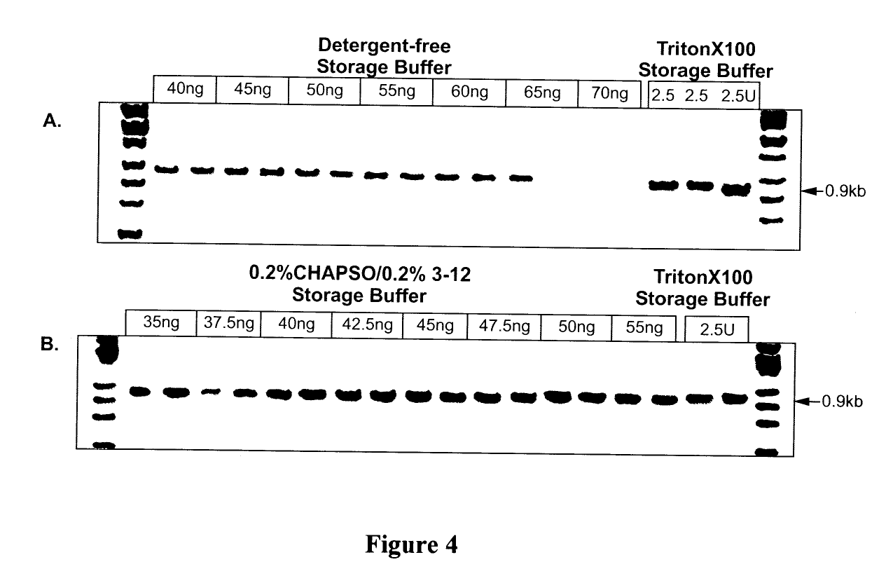

Zwitterionic detergents were also incorporated into enzyme storage buffers as seen in FIG. 4. For example, Pfu DNA polymerase was purified in the absence of detergent, and then diluted in storage buffers that lacked detergent (Panel A, lanes 2-15) or contained 0.2% each of the zwitterionic detergents, CHAPSO and Anzergent 3-12 (Panel B, lanes 2-17). When used in PCR with cloned Pfu PCR buffer (containing 0.1% Triton X100), Pfu samples prepared with zwitterionic detergents produced significantly higher yields than Pfu samples that were diluted and stored in the absence of CHAPSO and Anzergent 3-12.

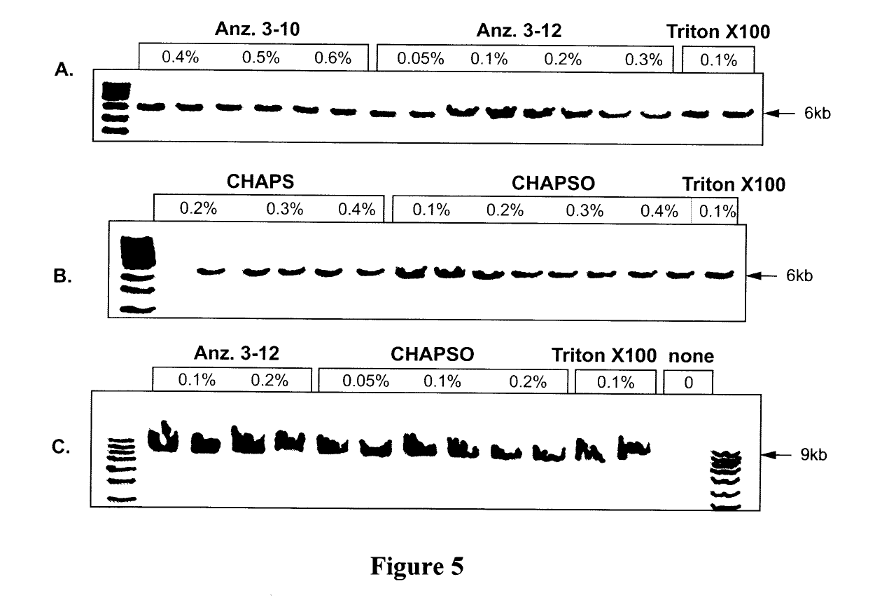

In addition to enhancing the storage stability of Pfu, zwitterionic detergents were also shown to increase yields when incorporated into PCR buffers (FIG. 5). PCR conducted in the buffer lacking detergent failed to generate product (Panel C, lane 14). In contrast, the addition of zwitterionic detergents dramatically improved product yields, and CHAPSO (0.05-0.2%; Panel B, lanes 8-15) and Anzergent 3-12 (0.1-0.2%; Panel A, lanes 8-15) were found to be somewhat more effective than CHAPS (Panel B, lanes 2-7) and Anzergent 3-10 (Panel A, lanes 2-7).

The detergents shown to enhance Pfu and Pfu fusion DNA polymerase activity include, without limitation, those listed in the following Table:

TABLE 2
Enhancing Detergents and Detergent Combinations

| Effective range | | Optimal range | |
|---|---|---|---|
| Detergent 1 | Detergent 2 | Detergent 1 | Detergent 2 |
| CHAPS (0.2-0.8%) | | CHAPS (0.2-0.4%) | |
| CHAPSO (0.1-0.8%) | | CHAPSO (0.15-0.35%) | |
| Anz. 3-10 (0.4-0.8%) | | Anz. 3-10 (0.4-0.8%) | |
| Anz. 3-12 (0.1-0.4%) | | Anz. 3-12 (0.1-0.2%) | |
| CHAPS (0.05, 0.1%) | Anz. 3-10 (0.1-0.5%) | CHAPS (0.05, 0.1%) | Anz. 3-10 (0.1-0.5%) |
| CHAPS (0.05%) | Anz. 3-12 (0.05-0.5%) | CHAPS (0.05%) | Anz. 3-12 (0.05-0.3%) |
| CHAPS (0.1%) | Anz. 3-12 (0.05-0.5%) | CHAPS (0.1%) | Anz. 3-12 (0.05-0.5%) |
| CHAPSO (0.1%) | Anz. 3-10 (0.05-0.5%) | CHAPSO (0.1%) | Anz. 3-10 (0.05-0.4%) |
| CHAPSO (0.05%, 0.1%) | Anz. 3-12 (0.05-0.5%) | CHAPSO (0.05, 0.1%) | Anz. 3-12 (O.05-0.4%) |
| CHAPS (0.05-0.5%) | CHAPSO (0.05-0.3%) | | |

### Example 3

### Using Zwitterionic Detergents to Stabilize Pfu Fusion Enzyme

Accelerated stability studies were performed to illustrate the stabilizing effects of zwitterionic detergents. Pfu fusion DNA polymerase was purified in the absence of detergents and then diluted to 28 ng/ul in storage buffer lacking detergent or storage buffers containing either conventional non-ionic detergent (0.1% Igepal/0.1% Triton X100) or various zwitterionic detergents. Protein samples were stored at −20° C. or were heated at 95° C. for varying lengths of time. Residual activity was assayed by amplifying a 0.9 kb genomic target in PCR in fusion DF-buffer supplemented with either 0.1% Triton X100 or 0.1% CHAPSO/0.1% Anzergent 3-12.

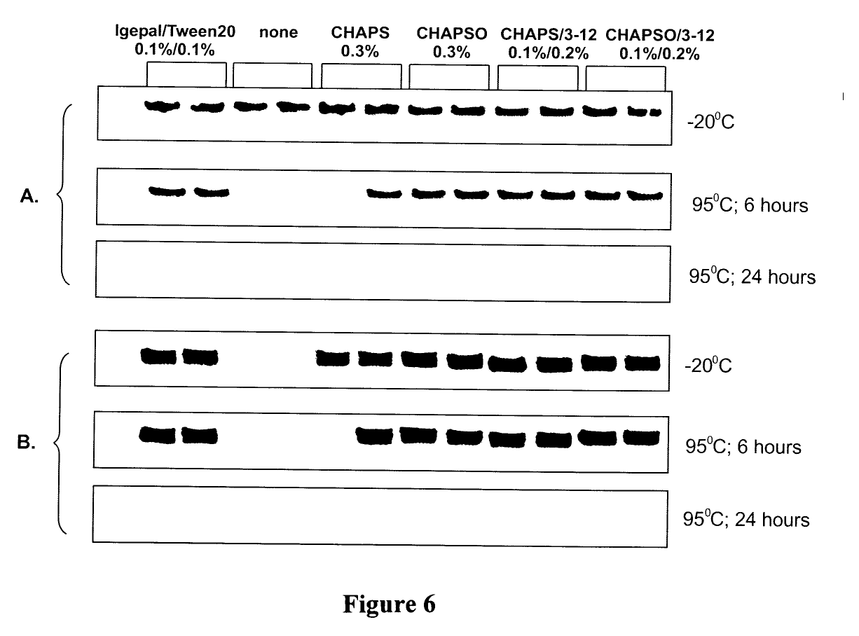

The results shown in FIG. 6 indicate that zwitterionic detergents CHAPS, CHAPSO, and 3-12 enhance the stability of Pfu fusion DNA polymerase. Pfu fusion DNA polymerase lost activity when heated at 95° C. in the absence of detergent (All panels, lanes 4 and 5). Compared to conventional non-ionic detergents (All panels, lanes 2 and 3), zwitterionic detergents appear to be equally effective in protecting Pfu fusion against activity losses when stored at −20° C. (Panels A and D, lanes 6-13) or when incubated at 95 ° C. for 6 hours (Panels B and E, lanes 6-13). After 24 hours at 95° C., protein samples stored in non-ionic and zwitterionic detergents were completely inactive (Panels C and F, lanes 4-13). Results were consistent whether amplification occurred in fusion DF-buffer supplemented with 0.1% Triton X100 (Panels A, B and C) or 0.1% CHAPSO/0.1% Anzergent 3-12 (Panels D, E and F).

### Example 4

### Using Zwitterionic Detergents to Enhance Pfu Fusion DNA Polymerase Activity in QPCR

QPCR reactions contained DNA or cDNA template, varying amounts of primer (see Table 3 below), 300 uM each dNTP, 4 ng/ul exo Pfu fusion, 6 ng/ul hot start IgG, 0.4ng/ul single-stranded DNA-binding protein, 1× Pfu fusion DF-buffer II (pH 9), 4% DMSO, and 8% glycerol. QPCR reactions were supplemented with 0.1% Triton X100 or zwitterionic detergent, and with 0.5× SYBR Green (Molecular Probes S-7567). Reactions were cycled on the MX3000P Real-Time PCR System using the following conditions: (1 cycle) 95° C. 5 min; (40 cycles) 95° C. 10 sec, 60° C. 30 sec.

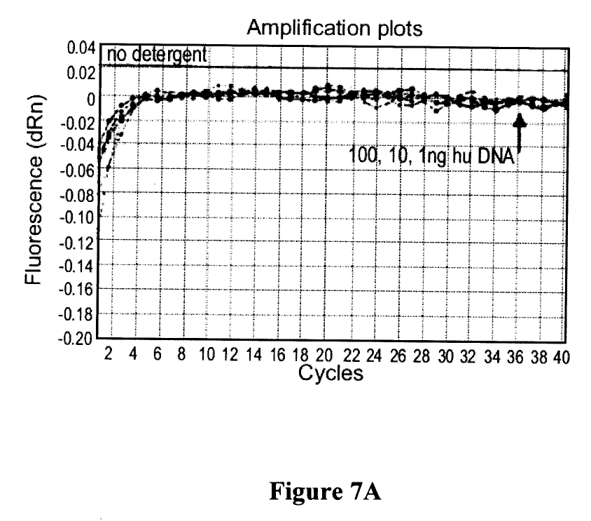

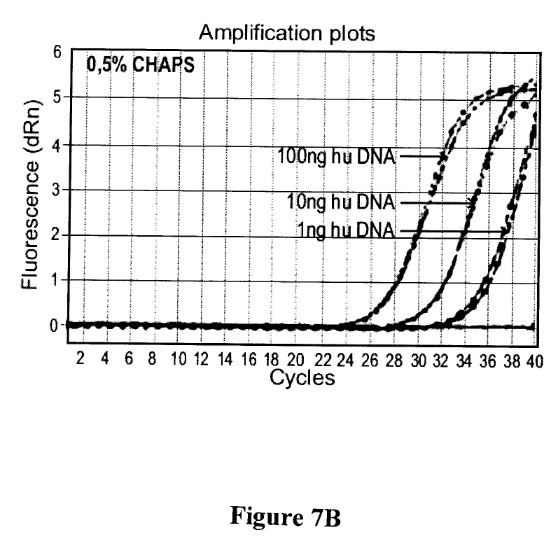

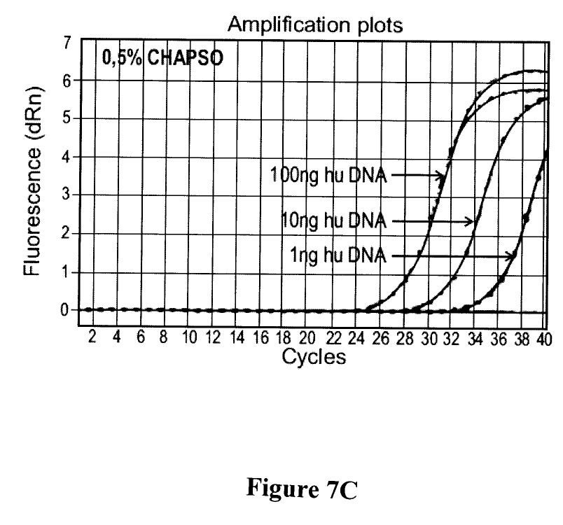

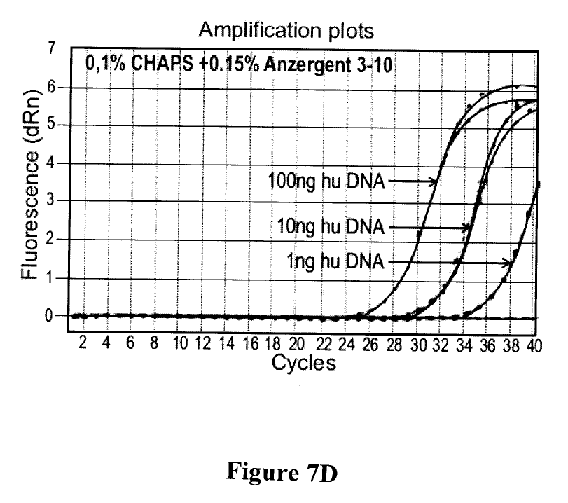

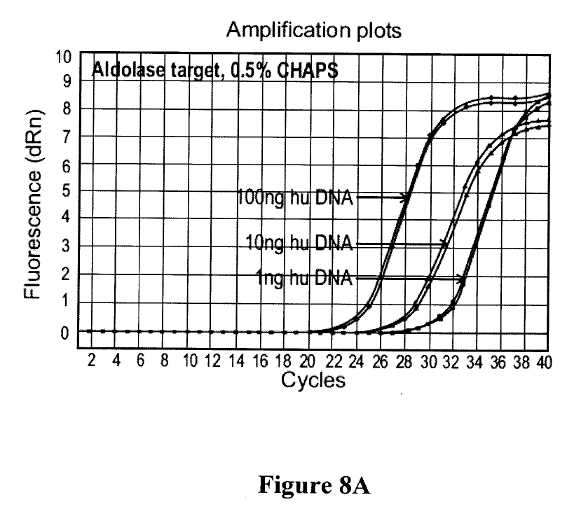

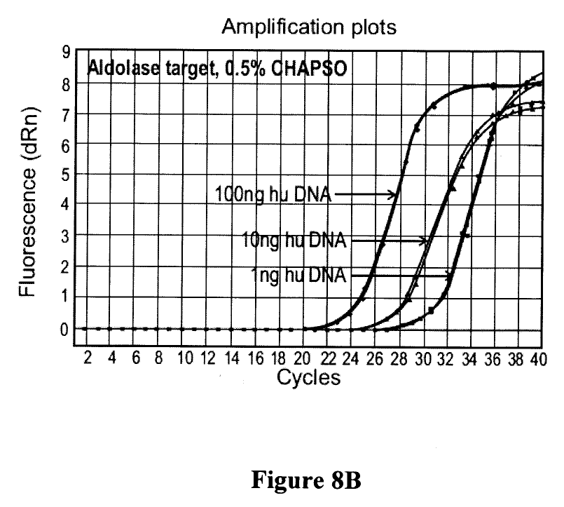

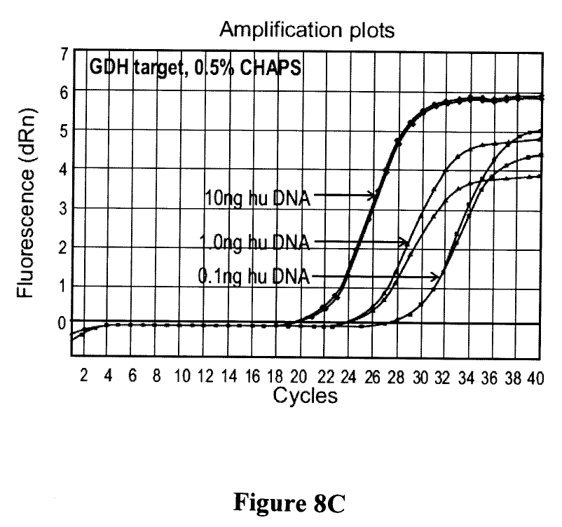

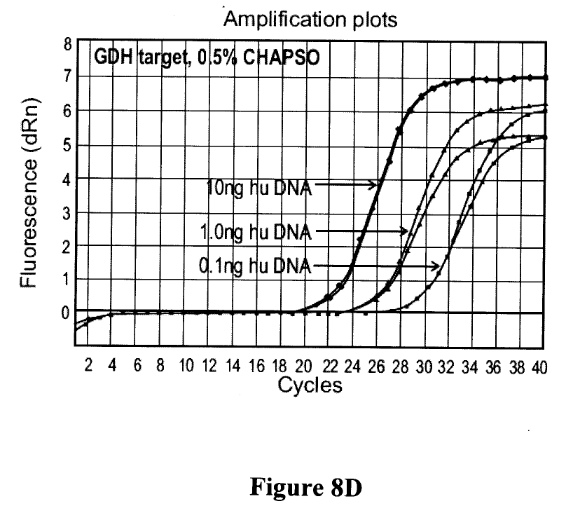

TABLE 3
QPCR Reaction Conditions Relating To FIGS. 7 and 8

| Target | | Primer | |
|---|---|---|---|
| (bp) | Template | Concentration | Primer Sequence |
| Hu gaucher | Human | 0.2 uM each | F:CCTGAGGGCTCCCAGAGAGTGG |
| (105) | genomic | | (SEQ ID NO: 6) |
| | DNA | | R:GGTTTAGCACGACCACAACAGC |
| | | | (SEQ ID NO: 7) |
| Hu | Human | 0.2 uM F | F:AGCCTAGCTCCAGTGCTTCTAGTA |
| aldolase | genomic | 0.3 uM R | (SEQ ID NO: 8) |
| (286) | DNA | | R:CTTTGGATGAGGAGCCGATATTG |
| | | | (SEQ ID NO: 9) |
| Hu GDH | Reverse- | 0.3 uM each | F: GATGGATCATGGCTGACTTC |
| (354) | transcribed | | (SEQ ID NO: 10) |
| | human RNA | | R AGCAAGCAACTGACTGCTCT |
| | | | (SEQ ID NO: 11) |

The results shown in FIGS. 7 and 8 demonstrate the enhancing activity of zwitterionic detergents in QPCR. Amplifications were conducted using QPCR Mastermixes (Stratagene catalog #600581) formulated with detergent-free Pfu fusion DNA polymerase, SYBR Green, and various zwitterionic detergents. As shown in FIG. 7, QPCRs conducted in the absence of detergent failed to generate product (Panel A). In contrast, amplifications performed in the presence of 0.5% CHAPS (Panel B) or CHAPSO (Panel C) appear comparable to those conducted in the presence of the conventional non-ionic detergent 0.1% Triton X100, with respect to amplification efficiency, total fluorescence, and Ct values. Combinations of zwitterionic detergents can be as effective or more effective than individual zwitterionic detergents. For example, the combination of 0.1% CHAPS and 0.15% Anzergent 3-10 is particularly effective in enhancing Pfu fusion activity in QPCR (Panel D). FIG. 8 demonstrates the enhancing activity of zwitterionic detergents when aldolase (Panels A and B) and GDH (Panels C and D) gene targets were amplified in PCR reactions supplemented with 0.5% CHAPS (Panels A and C) or 0.5% CHAPSO (Panels B and D). The addition of 0.5% CHAPS or 0.5% CHAPSO appeared favorable with respect to amplification efficiency, total fluorescence, and Ct values.

The following zwitterionic detergents or detergent combinations enhance QPCR amplifications conducted with Pfu fusion and SYBR Green detection:

TABLE 4
Enhancing Detergents And Detergent Combinations For SYBR Green QPCR

| Effective range | | Optimal range | |
|---|---|---|---|
| Detergent 1 | Detergent 2 | Detergent 1 | Detergent 2 |
| CHAPS (0.1-0.5%) | | CHAPS (0.5%) | |
| CHAPSO (0.1-0.75%) | | CHAPSO (0.2-0.5%) | |
| Anz. 3-10 (0.1-0.3%) | | Anz. 3-10 (0.2%) | |
| Anz. 3-12 (0.1-0.2%) | | Anz. 3-12 (0.15-0.2%) | |
| CHAPS (0.1%) | Anz. 3-10 (0.02-0.15%) | CHAPS (0.1%) | Anz. 3-10 (0.05-0.15%) |

### Example 5

### Using Non-Detergent Surfactant to Enhance Pfu DNA Polymerase Activity in Endpoint PCR

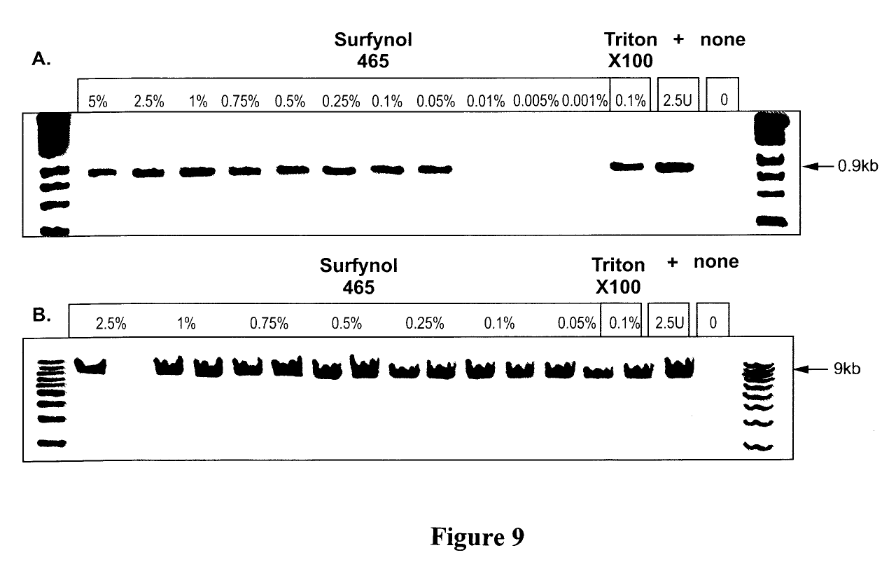

The results shown in FIG. 9 demonstrate the enhancing and/or stabilizing activity of Surfynol 465 on Pfu fusion (Panel A) and non-fusion (Panel B) DNA polymerases. PCRs were conducted using the conditions described in Example 2. Amplifications performed in the absence of non-ionic detergents generated high product yields when PCR reaction buffers were supplemented with 0.05% to 2.5% Surfynol 465 (Panel A, lanes 3-9 and Panel B, lanes 2-15). Yields were similar to those obtained using detergent-free PCR buffers that were further supplemented with 0.1% Triton X100 (Panel A, lanes 13 and 14; Panel B, lanes 16 and 17).

All patents, patent applications, and published references cited herein are hereby incorporated by reference in their entirety. While this invention has been particularly shown and described with references to preferred embodiments thereof, it will be understood by those skilled in the art that various changes in form and details may be made therein without departing from the scope of the invention encompassed by the appended claims.

1. A composition comprising at least one purified polymerase and at least one zwitterionic detergent or non-detergent surfactant.

2. The composition of claim 1, wherein said at least one purified polymerase is thermostable.

3. The composition of claim 2, wherein said thermostable purified polymerase is Pfu DNA polymerase, a thermostable DNA polymerase fusion protein, a Pfu DNA polymerase-Sso7 fusion polypeptide, or Taq DNA polymerase.

4. The composition of claim 1, wherein said at least one zwitterionic detergent or non-detergent surfactant is CHAPS, CHAPSO, n-Decyl-N,N-dimethyl-3-ammonio-1-propanesulfate (Anzergent/Zwittergent 3-10), n-Dodecyl-N,N-dimethyl-3-ammonio-1-propanesulfonate (Anzergent/Zwittergent 3-12), Surfynol 104, Surfynol 420, Surfynol 440, Surfynol 465, Surfynol 485, Surfynol 504, Surfynol PSA series, Surfynol SE series, Dynol 604, Surfynol DF series, Surfynol CT series, or Surfynol EP series.

5. The composition of claim 1, comprising a combination of two or more zwitterionic detergents or non-detergent surfactants.

6. The composition of claim 1, further comprising an oligonucleotide probe and a detectable label, wherein said detectable label is operatively coupled to said oligonucleotide probe.

7. The composition of claim 1, further comprising a labeled nucleotide.

8. The composition of claim 1, further comprising a fluorescent DNA binding dye, wherein said fluorescent DNA binding dye produces a detectable signal when bound to DNA.

9. A kit comprising a storage or reaction composition comprising a purified polymerase and at least one zwitterionic detergent or non-detergent surfactant.

10. The kit of claim 9, wherein the polymerase is thermostable.

11. The kit of claim 10, wherein said thermostable purified polymerase is Pfu DNA polymerase, a thermostable DNA polymerase fusion protein, a Pfu DNA polymerase-Sso7 fusion polypeptide, or Taq DNA polymerase.

12. A method for increasing the efficiency of a purified polymerase, the method comprising:
mixing a target nucleic acid with at least one purified polymerase, a primer, nucleoside-5′-triphosphates, and at least one zwitterionic detergent or non-detergent surfactant; and
allowing amplification of said target nucleic acid to occur by subjecting the mixture to a thermal cycle.

13. The method of claim 12, wherein said at least one purified polymerase is thermostable.

14. The method of claim 13, wherein said thermostable purified polymerase is Pfu DNA polymerase, a thermostable DNA polymerase fusion protein, a Pfu DNA polymerase-Sso7 fusion polypeptide, or Taq DNA polymerase.

15. The method of claim 12, wherein said at least one zwitterionic detergent or non-detergent surfactant is CHAPS, CHAPSO, n-Decyl-N,N-dimethyl-3-ammonio-1-propanesulfate (Anzergent/Zwittergent 3-10), n-Dodecyl-N,N-dimethyl-3-ammonio-1-propanesulfonate (Anzergent/Zwittergent 3-12), Surfynol 104, Surfynol 420, Surfynol 440, Surfynol 465, Surfynol 485, Surfynol 504, Surfynol PSA series, Surfynol SE series, Dynol 604, Surfynol DF series, Surfynol CT series, or Surfynol EP series.

16. The method of claim 12, further comprising mixing said target nucleic acid with a detectable label.

17. A method for detecting a target nucleic acid, the method comprising:
forming a reaction mixture comprising at least one purified polymerase, a primer, at least one zwitterionic detergent or non-detergent surfactant, nucleoside-5′-triphosphates, and a detectable label;
subjecting said reaction mixture to nucleic acid amplification reaction conditions which amplify said target nucleic acid; and
detecting a signal generated from said detectable label indicative of the presence and/or amount of said target in the sample.

18. The method of claim 17, wherein said at least one purified polymerase is thermostable.

19. The method of claim 18, wherein said thermostable purified polymerase is Pfu DNA polymerase, a thermostable DNA polymerase fusion protein, a Pfu DNA polymerase-Sso7 fusion polypeptide, or Taq DNA polymerase.

20. The method of claim 17, wherein said at least one zwitterionic detergent or non-detergent surfactant is CHAPS, CHAPSO, n-Decyl-N,N-dimethyl-3-ammonio-1-propanesulfate (Anzergent/Zwittergent 3-10), n-Dodecyl-N,N-dimethyl-3-ammonio-1-propanesulfonate (Anzergent/Zwittergent 3-12), Surfynol 104, Surfynol 420, Surfynol 440, Surfynol 465, Surfynol 485, Surfynol 504, Surfynol PSA series, Surfynol SE series, Dynol 604, Surfynol DF series, Surfynol CT series, or Surfynol EP series.
